SEQUENCE LISTING (2 sequences)

SEQ ID NO: 1 - DNA, 20 bp, Artificial Sequence
  Description of Artificial Sequence oligonucleotide primer
catacaggatgaaaagatgg

SEQ ID NO: 2 - DNA, 28 bp, Artificial Sequence
  Description of Artificial Sequence oligonucleotide primer
aattccatcttttcatcctgtatggtac

(19) United States
(12) Patent Application Publication
(10) Pub. No.: US 2002/0119440 A1
(43) Pub. Date: Aug. 29, 2002

(54) Peripheral nerve type sodium channels

(76) Inventors: Isamu Akiba, Kawabe-gun (JP); Tetsuo Seki, Kawanishi (JP); Seiichiro Nishimura, Takarazuka (JP); Edward Leon Barsoumian, Toyonaka (JP); Masaki Iizuka, Kawanishi (JP)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/993,224

(22) Filed: Nov. 6, 2001

Related U.S. Application Data
(60) Provisional application No. 60/249,932, filed on Nov. 17, 2000.

(30) Foreign Application Priority Data
Nov. 6, 2000 (DE) DE 100 54 878.4

(51) Int. Cl. C12Q 1/00; C12P 21/02; C12N 5/08; A61K 38/17

(52) U.S. Cl. 435/4; 435/69.1; 435/368; 435/320.1; 514/2

(57) ABSTRACT

The present invention provides cell lines expressing one or more recombinant peripheral nerve (PN) sodium channels, preferably a human PN sodium channel. The invention also provides methods for preparing said cell lines. The invention also pertains to the use of said cell lines for the identification of PN sodium channel agonists, antagonists or modulators and methods for the identification of said agonists, antagonists or modulators. The invention also provides pharmaceutical compositions comprising said agonists, antagonists or modulators. The invention also is concerned with the use of agonists, antagonists or modulators in the manufacture of a medicament for the treatment of chronic pain disorders.

### RELATED APPLICATION

The benefit of prior U.S. provisional application Ser. No. 60/249,932, filed Nov. 17, 2000 is hereby claimed.

### TECHNICAL FIELD OF THE INVENTION

The present invention belongs to the field of neurological research and in particular ionic channels and peripheral nerve channels.

### BACKGROUND

Voltage-gated sodium channels have been known to underlie axonal and somatic action potentials and amplify synaptic potentials in nerves. Several lines of evidence suggest that voltage-gated sodium channels located in peripheral sensory neurons may play an important role not only in the initial injury discharge but also in spontaneous, ongoing and stimulus-evoked pain and dysesthesia characteristic of many types of peripheral neuropathies (Waxman, et al, Proc. Natl. Acad. Sci. USA (1999) 96, 7635-7639; Mao J. Chen L L. Pain, (2000) 87, 7-17). The clinical effectiveness of agents that act primarily through a common use-dependent block of sodium channels, such as local anesthetics, antiarrhythmics and anticonvulsants, has been reported in the treatment of many neuropathic pain (Mao, J, Chen L L. (2000) Pain, 87, 7-17; Galer B S, et al (1996) J. Pain Symptom. Manage., 12, 161-167; McQuay H, et al (1995) Br. Med. J., 311, 1047-1052). However, the analgesic potential of these agents has been limited due to the numerous adverse side-effects including cardiotoxicity and CNS-related signs.

Recent studies have revealed novel tetrodotoxin(TTX)-sensitive and TTX-resistant sodium channels, the peripheral nerve type PN1 and PN3, respectively, (Klugbauer N, et al (1995) EMBO J., 14, 1084-1090; Akopian A N, et al (1996) Nature, 379, 257-262; Sangameswaran L, et al (1996) J. Biol. Chem., 271, 5953-5956; Toledo-Aral J J, et al (1997) Proc. Natl. Acad. Sci. USA, 94, 1527-1532; Sangameswaran L, et al (1997) J. Biol. Chem., 272, 14805-14809; Rabert D K, et al (1998) Pain, 78, 107-114). They are expressed at high levels throughout the peripheral nerve system, especially sensory C- and A-fibers, and contribute to nociceptive transduction. The expression of both channels is induced/enhanced by nerve growth factor (NGF) in in vitro and in vivo studies, which is released during inflammation and nerve injury (Toledo-Aral J J, et al (1995) Neuron, 14, 607-611; Black J A, et al (1997) NeuroReport, 8, 2331-2335). In the carageenan inflammatory pain model in the rat, it has been reported that PN3 mRNA levels and TTX-resistant sodium currents are significantly increased in dorsal root ganglion neurons projecting to the inflamed limb (Waxman S G, et al (1999) Proc. Natl. Acad. Sci. USA, 96, 7635-7639). These observations suggest that the peripheral nerve type sodium channels are involved in nociceptive transduction in inflammatory and neuropathic pains.

Investigations into the functional properties of these sodium channels have been carried out so far by transient expression in Xenopus oocytes (Akopian A N, et al (1996) Nature, 379, 257-262; Sangameswaran L, et al (1996) J. Biol. Chem., 271, 5953-5956; Sangameswaran L, et al (1997) J. Biol. Chem., 272, 14805-14809; Rabert D K, et al (1998) Pain, 78, 107-114) or in mammalian cell lines (Klugbauer N, et al (1995) EMBO J., 14, 1084-1090; Tate S, et al (1998) Nat. Neurosci., 1, 653-655). There is an unmet need in the art to find substances capable of selectively blocking peripheral nerve channels such as PN1 and PN3. Thus, the problem underlying the present invention is to provide the tools for finding such substances and the substances themselves.

### SUMMARY OF THE INVENTION

The above-captioned technical problem is solved by the embodiments characterized in the claims and the description. The before-mentioned disadvantages in the art are overcome by the claims and the description of the present invention.

The present invention provides cell lines expressing one or more recombinant peripheral nerve (PN) sodium channels, preferably a human PN sodium channel. The invention also provides methods for preparing said cell lines. The invention furthermore pertains to the use of said cell lines for the identification of PN sodium channel agonists, antagonists or modulators and methods for the identification of said agonists, antagonists or modulators. The invention also provides pharmaceutical compositions comprising said agonists, antagonists or modulators. The invention furthermore is concerned with the use of agonists, antagonists or modulators in the manufacture of a medicament for the treatment of neurological disorders.

### BRIEF DESCRIPTION OF THE FIGURES

FIG. 1

Northern blot analysis of RNA from stable transfectants using cDNA probes for human PN1 and PN3 sodium channels.

Expression of the PN1 transcript in hPN1 (#21-43) cells (A) and the PN3 transcript in hPN3 (#2) cells (B) was investigated in the presence of tetracycline, or at the indicated time points (24 hr-72 hr) after tetracycline removal from the culture medium. The migration positions of RNA size markers are shown on the left.

FIG. 2

Electrophysiological properties of human PN1 sodium channel expressed in hPN1 (#21-43) cells.

(A) Representative current traces evoked by 10-ms test pulses from −60 mV to 70 mV by a 10 mV step. Holding potential −100 mV. (B) The peak current amplitude-voltage relationship. Each point represents the mean±SE of 7 cells. (C) Dose-response curve of TTX. Each point represents the mean±SE of 5 cells.

FIG. 3

Electrophysiological properties of human PN3 sodium channel expressed in hPN3 (#2) cells. (A) Representative current traces evoked by 10-ms test pulses from −60 mV to 70 mV by a 10 mV step in the presence of 1M TTX. Holding potential −100 mV. (B) The peak current amplitude-voltage relationship in the presence of 1 M TTX. Each point represents the mean ±SE of 12 cells.

### DETAILED DESCRIPTION OF THE INVENTION

Before the embodiments of the present invention, it must be noted that as used herein and in the appended claims, the singular forms “a”, “an”, and “the” include plural reference unless the context clearly dictates otherwise. Thus, for example, reference to “a peripheral nerve type sodium channel” includes a plurality of such peripheral nerve type sodium channels, reference to the “cell” is a reference to one or more cells and equivalents thereof known to those skilled in the art, and so forth. By “cell line” it is meant a clone of a primary cell that is capable of stable growth in vitro for many generations. By “clone” it is meant a population of cells derived from a single cell or common ancestor by mitosis. By “variant” it is meant a sequence, such as a polynucleotide or polypeptide, that differs from another sequence, but retains essential properties thereof, that is, properties for which the sequence is utilized in its application (e.g., promoting expression, cleaving a bond, etc.). For example, a variant of a polynucleotide may differ in nucleotide sequence by one or more substitutions, additions, and deletions, from the reference polynucleotide. By “variant” it is also meant to include fragments of a full length sequence that retain essential properties thereof. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

The present invention provides a new cell line stably expressing one or more recombinant peripheral nerve (PN) sodium channels on its surface.

The term “peripheral nerve type sodium channel”, “peripheral nerve sodium channel” or “PN sodium channel” (both terms are used interchangeable) as used herein relates to peripherally expressed sodium channels such as tetrodotoxin(TTX)-sensitive PN1 and TTX-resistant PN3. They may be derived from any vertebrate species, preferably from mammalian species such as rat, mouse, hamster, pig, cattle, deer, horse and most preferably human. Primary neuronal cells, as they are post-mitotic cells and have to be freshly prepared for every experiment, are not very useful in many research applications and in particular not in industrial applications such as HTS.

There are no reports on the establishment of stable cell lines expressing sodium channels due to difficulties in constitutive expression under physiologically unregulated conditions. The present invention has overcome this problem in the art. The present invention now provides cell lines stably expressing PN sodium channels which is a research tool most closely simulating the natural situation and thus overcomes the disadvantages in the prior art where only primary neuronal cells and transiently transfected cell lines are available.

Cells which have been “stably transformed” have recombinant DNA incorporated into their genomic DNA. Such stably incorporated DNA is retained by the transformed cells because it is introduced into the cells with a selection agent which forces retention when the cells are grown in a selection medium. The present invention preferably employs mammalian cell lines that have been stably transformed.

Said recombinant DNA is under control of a transcriptional initiation region (or promoter) which may be a constitutive promoter or an inducible or developmentally regulated promoter. Of particular interest but not exclusive are the constitutive promoters of the human cytomegalovirus (CMV) and Rous sarcoma virus (RSV), as well as the Simian virus 40 and Herpes simplex promoters. Useful inducible promoters include antibiotic resistant promoters, heat-shock promoters, the hormone-inducible mammary tumor promoter and the metallothionein promoter.

A suitable cell or cell line, preferably a eukaryotic cell or a cell line, to be transformed with nucleic acid constructs to express a recombinant PN sodium channel on its cell surface may be any cell or cell line known to the expert in the field, in particular cells or cell lines used in neurological and neurobiology research. Examples of such cells or cell lines useful for producing the transformed cell lines of the invention include mammalian cells or cell lines (e.g. the cell lines human embryonic kidney (HEK) 293, BHK, GH3, H4, U373, NT2, PCI2, COS, CHO, Ltk-, fibroblasts, myelomas, neuroblastomas, hybridomas, oocytes, embryonic stem cells), insect cell lines (e.g., using baculovirus vectors such as pPbac or pMbac (Stratagene, La Jolla, USA)), yeast (e.g., Pichia pastoris or using yeast expression vectors such as pYESHIS (Invitrogen, San Diego, USA)), and fungi.

In a more preferred embodiment, the invention relates to a cell line as described, wherein said sodium channel comprises the peripheral nerve type sodium channel 1 (PN1).

In another more preferred embodiment, the invention relates to a cell line as described, wherein said sodium channel comprises the peripheral nerve type sodium channel 3 (PN3).

In another more preferred embodiment, the invention relates to a cell line as described, wherein said sodium channel comprises a protein of the amino acid sequence as in Genbank accession number X82835 or a functional derivative or a fragment thereof.

In another more preferred embodiment, the invention relates to a cell line as described, wherein said sodium channel comprises a protein of the amino acid sequence as in Genbank accession number AF117907 or a functional derivative or a fragment thereof.

In a preferred embodiment, the invention relates to a cell line as described, wherein said cell line expresses a recombinant human peripheral nerve type sodium channel. Said cell line stably expressing a human PN channel can be a more precise, species-targeted tool for the identification of novel drugs useful in the therapy of chronic pain disorders.

In another more preferred embodiment, the invention relates to a cell line as described which is derived from the CHO cell line.

In another important embodiment, the invention relates to a CHO cell line stably expressing a recombinant human PN1 sodium channel on its cell surface.

In another important embodiment, the invention relates to a CHO cell line stably expressing a recombinant human PN3 sodium channel on its cell surface. Said cell lines of the present invention expressing a human PN sodium channel such as PN1 or PN3 (exemplified in example 1) can overcome the disadvantages in the prior art and can be a more precise, species-targeted tool for the identification of novel drugs useful in the therapy of chronic pain disorders.

Surprisingly, said cell lines of the present invention possess functional PN sodium channels comparable to channels on primary neuronal cells on the basis of electrophysiological and pharmacological properties; Example 1.

Another preferred embodiment of the present invention is a nucleic acid encoding a recombinant human PN sodium channel or recombinant human PN sodium channels.

Another more preferred embodiment of the present invention is a nucleic acid according to the invention, wherein said nucleic acid is characterized by a nucleic acid sequence comprising the sequence in Genbank accession number X82835 or a functional derivative or a fragment thereof or a variant due to the degenerate code.

Another more preferred embodiment of the present invention is a nucleic acid according to the invention, wherein said nucleic acid is characterized by a nucleic acid sequence comprising the sequence in Genbank accession number AF117907 or a functional derivative or a fragment thereof or a variant due to the degenerate code.

Another important aspect of the present invention is a method for preparing a cell line as described supra comprising the construction of an expression vector which comprises DNA specific for a PN sodium channel, the transfection of a suitable cell line with said expression vector and the screening of said cell line for stable transfection employing a suitable selection agent.

For the purpose of the present invention, a nucleic acid encoding a PN sodium channel can be obtained from any cDNA library prepared from tissue believed to possess the PN sodium channel mRNA and to express it at a detectable level. For example, a human peripheral and central neuronal tissue-derived cDNA library, such as that described in the examples, is a good source of a PN sodium channel cDNA. The PN sodium channel genes can also be obtained from a genomic library, such as a human genomic cosmid library. The cDNA encoding the PN sodium channel may be then used for the ligation into appropriate vectors and transformation of appropriate cells or cell lines with said vectors (for a non-limiting example of the invention, see Example 1).

Methods of producing appropriate recombinant vectors, transforming cells with those recombinant vectors, and identifying transformants are well known in the art and are only briefly reviewed here (see, for example, Sambrook et al. (1989). Molecular Cloning: A Laboratory Manual, 2^{nd} ed., Cold Spring Harbor Laboratory Press, Cold Spring Harbor, N.Y.).

Suitable vectors comprise plasmids, viruses (including phages) and integratable DNA fragments (i.e., integratable into the host genome by recombination). In the present specification, “vector” is generic to “plasmid”; but plasmids are the most commonly used form of vectors at present. However, all other forms of vectors which serve an equivalent function and which are, or become, known in the art are suitable for use herein. Suitable vectors will contain replicon and control sequences which are derived from species compatible with the intended expression host.

Suitable host cells are any prokaryotes, yeasts or higher eukaryotic cells which have been transformed or transfected with the nucleic acids of the present invention so as to cause clonal propagation of those nucleic acids and/or expression of the proteins or peptides encoded thereby. Such cells or cell lines will have utility both in the propagation and production of the nucleic acids and proteins of the present invention but also, as further described herein, as model systems for diagnostic and therapeutic assays. As used herein, the term “transformed cell” is intended to embrace any cell, or the descendant of any cell, into which has been introduced any of the nucleic acids of the invention, whether by transformation, transfection, infection, or other means.

Prokaryotic cells useful for producing the transformed cells of the invention include members of the bacterial genera Escherichia (e.g., E. coli), Pseudomonas (e.g., P. aeruginosa), and Bacillus (e.g., B. subtillus, B. stearothermophilus), as well as many others well known and frequently used in the art. Prokaryotic cells are particularly useful for the production of large quantities of the receptor proteins of the invention (e.g. normal or mutant PN sodium channels or subunits thereof, fragments of the said receptors or subunits, fusion proteins of said receptors or subunits). Bacterial cells (e.g., E. coli) may be used with a variety of expression vector systems including, for example, plasmids with the T7 RNA polymerase/promoter system, bacteriophage X regulatory sequences, or M13 Phage mGP-12. Bacterial hosts may also be transformed with fusion protein vectors which create, for example, lacZ, trpE, maltose-binding protein, poly-His tags, or glutathione-S-transferase fusion proteins. All of these, as well as many other prokaryotic expression systems, are well known in the art and are widely commercially available (e.g., pGEX-27 (Amrad, USA) for GST fusions).

Eukaryotic cells and cell lines useful for producing the transformed cells of the invention are described infra.

To accomplish expression in eukaryotic cells, a wide variety of vectors have been developed and are commercially available which allow inducible (e.g., LacSwitch expression vectors, Stratagene, La Jolla, Calif.) or cognate (e.g., pcDNA3 vectors, Invitrogen, San Diego, USA) nucleotide sequence expression of PN sodium channel subunits or entire channels under the regulation of an artificial promoter element. Such promoter elements are often derived from CMV or SV40 viral genes, although other strong promoter elements which are active in eukaryotic cells can also be employed to induce transcription of expression of PN sodium channel subunits or entire channel nucleotide sequences. Typically, these vectors also contain an artificial polyadenylation sequence and 3′ UTR which can also be derived from exogenous viral gene sequences or from other eukaryotic genes. Furthermore, in some constructs, artificial, non-coding, spliceable introns and exons are included in the vector to enhance expression of the nucleotide sequence of interest (in this case, PN sodium channel nucleotide sequences). These expression systems are commonly available from commercial sources and are typified by vectors such as pcDNA3 and pZeoSV (Invitrogen, San Diego, USA). Innumerable commercially-available as well as custom-designed expression vectors are available from commercial sources to allow expression of any desired expression of PN sodium channel transcript in more or less any desired cell type, either constitutively or after exposure to a certain exogenous stimulus (e.g., withdrawal of tetracycline or exposure to IPTG).

Recombinant vectors may be introduced into the recipient or “host” cells by various methods well known in the art including, but not limited to, calcium phosphate transfection, strontium phosphate transfection, DEAE dextran transfection, electroporation, lipofection (e.g., Dosper Liposomal transfection reagent, Boehringer Mannheim, Germany), microinjection, ballistic insertion on micro-beads, protoplast fusion or, for viral or phage vectors, by infection with the recombinant virus or phage.

Thus, stable transformation of a mammalian cell line may be accomplished by using standard methods to co-transfect the cells with one or several recombinant vectors comprising nucleic acid molecules encoding a PN sodium channel according to the present invention and with a second vector which confers resistance to a selection agent such as an antibiotic, e.g. G418 (neomycin) or Zeocin. Alternatively, transformation can be carried out with a single vector containing both the genetic control element and the selection agent gene. Recombinant retroviral vectors can also contain a selection agent gene to produce stable transformation. To co-transfect cells to express PN sodium channels according to the present invention, a different selection agent for every PN sodium channel cDNA may be used.

Thus, another important aspect of the present invention is a method as described, wherein said cell line is co-transfected with two or more of said expression vectors each of which comprises DNA specific for a different PN sodium channel.

The cell lines according to the present invention, preferably the cell lines expressing the human PN1 or PN3 channels, constitute valuable tools for the artisan. Said cell lines of the present invention are more precise, species-targeted tools for identification and characterization of new chronic pain modulators.

Thus, another important embodiment of the present invention is the use of a cell line according to the description supra for the identification of PN sodium channel agonists.

Yet another important embodiment of the present invention is the use of a cell line according to the description supra for the identification of PN sodium channel antagonists or modulators.

The term “agonist”, as used herein, refers to a substance or signal, that activates PN sodium channel function; and the terms “antagonist” or “modulator” refer to a substance that interferes with PN sodium channel function.

Agonists include, but are not limited to chemical compounds such as small organic molecules, proteins, nucleic acids, carbohydrates, or any other molecules which bind to PN sodium channels. The terms “antagonist” or “modulator” as used herein, refer to a molecule which, when bound to PN sodium channels, blocks or modulates the biological or neurological activity of PN sodium channels e.g. by blocking the activation of the PN sodium channel through agonists. Modulators as used herein refer e.g., to allosteric modulators. Antagonists or modulators include competitive and non-competitive antagonists or modulators. A competitive antagonist (or competitive blocker) interacts with or near the site specific for the agonist (e.g., an PN sodium channel ligand) for the same or closely situated site. A non-competitive antagonist or modulator or blocker inactivates the functioning of the receptor by interacting with a site other than the site that interacts with the agonist. Antagonists or modulators include, but are not limited to chemical compounds such as small organic molecules, proteins, nucleic acids, carbohydrates, or any other molecules which bind to PN sodium channels.

Yet another important embodiment of the present invention is the use of a cell line according to the description supra in a high throughput screening (HTS) format. HTS relates to an experimental setup wherein a large number of compounds is tested simultaneously. Preferably, said HTS setup may be carried out in microplates, may be partially or fully automated and may be linked to electronic devices such as computers for data storage, analysis, and interpretation using bioinformatics. Preferably, said automation may involve robots capable of handling large numbers of microplates and capable of carrying out several thousand tests per day. Preferably, a test compound which shows a desired agonist, antagonist or modulator function in a cell-free system will also be tested in a cell-based system using a cell line according to the present invention. The term HTS also comprises ultra high throughput screening formats (UHTS). Preferably, said UHTS formats may be carried out using 384- or 1536-well microplates, sub-microliter or sub-nanoliter pipettors, improved plate readers and procedures to deal with evaporation. HTS methods are described e.g. in U.S. Pat. No. 5,876,946 or U.S. Pat. No. 5,902,732 . The expert in the field can adapt the method described below to a HTS or UHTS format without the need of carrying out an inventive step.

Yet another important embodiment of the present invention is the use of a cell line according to the description supra in an electrophysiological assay with an agonist, antagonist or modulator of a PN sodium channel wherein membrane currents are measured. Electrophysiological assays are known to the artisan and are disclosed in e.g. Hille, B., 1984; Ionic channels of excitable membranes. Sinauer Associates Inc., Sunderland, USA. A non-limiting example of such an assay is disclosed in Example 1.

Yet another important embodiment of the present invention is the use of a cell line according to the description supra in a receptor-binding assay (see description infra).

Yet another important embodiment of the present invention is the use of a cell line according to the description supra in a colorimetric or fluorometric assay wherein the intracellular quantity of a monovalent ion is measured.

In a further important aspect, the present invention relates to a method for the identification of an agonist, antagonist or modulator of a PN sodium channel comprising the incubation of a cell line as described supra with a test substance.

In another important aspect, the invention relates to a method as described, further comprising the measurement of a membrane current and the comparison of said membrane current with the membrane current obtained for said cell line after incubation with a known control or in the absence of the test substance.

As understood by those skilled in the art, methods for the identification of an agonist, antagonist or modulator of a PN sodium channel generally require comparison to a control. One type of a “control” cell or “control” culture is a cell or culture that is treated substantially the same as the cell or culture exposed to the test compound, except the control culture is not exposed to test compound. For example, in methods that use voltage clamp electrophysiological procedures, the same cell can be tested in the presence and absence of test compound, by merely changing the external solution bathing the cell. Another type of “control” cell or “control” culture may be a cell or a culture of cells which are identical to the transfected cells, except the cells employed for the control culture do not express the PN sodium channels expressed in the stably transfected cells according to the invention. In this situation, the response of test cell to test compound is compared to the response (or lack of response) of receptor-negative (control) cell to test compound, when cells or cultures of each type of cell are exposed to substantially the same reaction conditions in the presence of compound being assayed. The above-mentioned known agonists, antagonists or modulators may also be used as a control for the methods of the present invention. In this case, cells or cultures of the cell lines according to the invention are incubated with the test substance. In a parallel experiment, cells or cultures of said cell lines are incubated with a known agonist, antagonist or modulator and both responses are compared.

Said methods according to the invention may be biochemical, molecular biology or immunological methods. Biochemical or molecular biology methods are known to the expert in the field and include, but are not limited to: reporter gene assays such as β-gal-, CAT-, SEAP- GFP-, BFP- or luciferase-assays, polymerase-chain reaction (PCR), RT-PCR, Northern- or Southern-blots which are published e.g. in: Sambrook et al.(1989) Molecular Cloning: A Laboratory Manual, 2^{nd} ed., Cold Spring Harbor Laboratory Press, Cold Spring Harbor, N.Y. and Bertram, S. and Gassen, H. G. Gentechnische Methoden, G. Fischer Verlag, Stuttgart, N.Y., 1991). Immunological methods are known to the expert in the field and include, but are not limited to ELISAs (enzyme-linked immuno-sorbent assay) or Sandwich-ELISAs, dot-blots, immunoblots, radioimmunoassays (Radioimmunoassay RJA), diffusion-based Ouchterlony tests, rocket immunofluorescent assays or Western-blots. Examples for immunological methods are e.g. described in: An Introduction to Radioimmunoassay and Related Techniques, Elsevier Science Publishers, Amsterdam. The Netherlands (1986); Bullock et al, Techniques in Immunocytochemistry, Academic Press, Orlando, Fla. Vol. 1 (1982), Vol. 2 (1983), Vol. 3 (1985); Tijssen, Practice and Theory of Enzyme Immunoassays: Laboratory Techniques in Biochemistry and Molecular Biology, Elsevier Science Publishers, Amsterdam, The Netherlands (1985).

Furthermore, the methods according to the present invention preferably include electrophysiological methods or assays as described supra, e.g. a patch-clamp method (see also e.g. Mayer M L, Vyklicky L Jr, Westbrook G L (1989); Modulation of excitatory amino acid receptors by group IIB metal cations in cultured mouse hippocampal neurones. J Physiol 415, 329-350). The artisan knows numerous methods which can be used for the identification of agonists, antagonists or modulators of PN sodium channels and can adapt them without carrying out an inventive step to include a cell line according to the present invention (see e.g. Hille, B., 1984; Ionic channels of excitable membranes. Sinauer Associates Inc., Sunderland, USA.). Test compounds, e.g. libraries containing small organic molecules, are widely available from commercial sources and can be included in the methods according to the present invention.

In another preferred aspect, the invention relates to a method as described, wherein said method is characterized in that a cell line as described supra is incubated with a known agonist which is coupled to a reporter, said cell line is incubated with a test substance, and the displacement of the agonist coupled to the reporter by the test substance is measured. All above-mentioned known agonists can be used in this preferred method and can be coupled to a reporter without inventive step. A reporter as used herein may be any reporter known to the artisan and will depend upon the type of method used. Examples of reporters that can be used include, e.g., radiolabels such as ³²P, ¹²⁵I, ³H and ¹⁴C; fluorescent reporters such as fluorescein and its derivatives, rhodamine and its derivatives, dansyl and umbelliferone and chemiluminescers such as the various luciferin compounds. For example, this method may be a receptor-binding assay, wherein e.g. the known agonist is coupled to the reporter tritium [³H] and the displacement of [³H]-agonist is measured.

In another preferred aspect, the invention relates to a method as described, wherein said method is characterized in that a cell line as described supra is incubated with a test substance, the intracellular quantity of a monovalent cation is measured, said quantity of the intracellular monovalent cation (e.g. sodium, potassium) is compared with the quantity of the intracellular monovalent ion measured for said cell line after incubation with a known control or in the absence of the test substance. The above-mentioned known agonists, antagonists or modulators can be used as a control for this preferred method of the present invention. Said method may be an electrophysiological method wherein e.g. electrophysiological changes observed following activation or inhibiton of the PN sodium channels are measured.

In a further preferred aspect, the present invention relates to a method as described, wherein said method is a high throughput screening assay (HTS; see description supra).

Yet another important embodiment of the invention is an agonist, antagonist or modulator of a PN sodium channel identifiable with a method according to the invention. Examples for PN sodium channel antagonists or modulators are lidocaine, mexiletine and TTX which may serve as control for the methods according to the invention. Preferably, said agonist, antagonist or modulator according to the invention is not selected from the group of lidocaine, mexiletine and TTX.

Another preferred embodiment of the present invention is a pharmaceutical composition comprising an agonist, antagonist or modulator of a PN sodium channel identifiable with a method as described supra and a pharmaceutically acceptable carrier. Preferably, said pharmaceutical composition comprises an agonist, antagonist or modulator according to the invention which is not selected from the group of lidocaine, mexiletine and TTX.

A pharmaceutically acceptable carrier can contain physiologically acceptable compounds that act, for example, to stabilize or to increase the absorption of a PN sodium channel agonist, antagonist or modulator. Such physiologically acceptable compounds include, for example, carbohydrates, such as glucose, sucrose or dextran, antioxidants, such as ascorbic acid or glutathione, chelating agents, low molecular weight proteins or other stabilizers or excipients (see also e.g. Remington's Pharmaceutical Sciences (1990). 18th ed. Mack Publ., Easton). One skilled in the art would know that the choice of a pharmaceutically acceptable carrier, including a physiologically acceptable compound, depends, for example, on the route of administration of the composition.

Another preferred embodiment of the present invention is the use of an agonist or antagonist of a PN sodium channel identifiable with a method according to the present invention in the manufacture of a medicament for the treatment of chronic pain disorders. Preferably, said use of an agonist, antagonist or modulator according to the invention comprises an agonist, antagonist or modulator which is not selected from the group of lidocaine, mexiletine and TTX. Chronic pain disorders include, but are not limited to disorders involving the development and maintenance of chronic pain such as chronic inflammatory diseases, neuropathic pain, diabetic neuropathy, cancer and the like.

The following example serves to further illustrate the present invention; but the same should not be construed as limiting the scope of the invention disclosed herein.

### EXAMPLE 1

### Establishment of Stable Transfectants Expressing the Human PN1 and PN3 Sodium Channels

### Materials and Methods

1. Cloning and Expression Constructs

1-1. Construction of Expression Plasmids Containing the Human PN 1 cDNA

Complementary DNA encoding the human PN1 was derived from RT-PCR. One μg human adrenal gland poly(A)⁺ RNA (Clontech, Tokyo, Japan) was subjected to Moloney's murine leukemia virus RNase H-reverse transcriptase (Toyobo, Osaka, Japan) with random primers. The synthesized first strand cDNA was amplified with a DNA Thermal Cycler (Perkin-Elmer Corp., Norwalk, Conn.), according to the manufacturer's specifications (TaKaRa LA PCR kit, Shiga, Japan).

Primer pairs were synthetic 25-nucleotide oligomers as follows;

1-25 (ST; upstream primer) and 2266-2290 (B; downstream primer),

2276-2300 (A; upstream primer) and 3914-3938 (D; downstream primer),

3902-3926 (C; upstream primer) and 5120-5144 (F; downstream primer), and

5109-5133 (E; upstream primer) and 6091-6115 (ED; downstream primer).

The numbering of nucleotides is according to the sequence derived from GenBank (accession number; X82835). The reverse transcriptase PCR products of the above primer pairs were ligated into pCR™ 2.1 vector (Invitrogen, Carlsbad, Calif.) to yield PHPN1(ST/B)/pCR™2.1, pHPN1(A/D)/pCR™2.1, pHPN1 (C/F)/pCR™2.1 and pHPN1 (E/ED)/pCR™2.1, respectively. The plasmid pZeoSV (Invitrogen, San Diego, Calif.) was cleaved by SpeI and XhoI, and ligated with the 1.6 kb XhoI(vector)/SpeI(vector) fragment from PHPN1 (A/D)/pCR™2.1 to yield pHPN1(A/D)/pZeoSV. The pHPN1(A/D)/pZeoSV was cleaved by DraI(2283) and NotI(vector), and ligated with the 2.3 kb NotI(vector)/DraI(2282) fragment from pHPNI(ST/B)/pCR™2.1 to yield pHPN1 (ST/D)/pZeoSV. The plasmid pBluescriptII KS(−) (Stratagene, La Jolla, Calif.) was cleaved by SpeI and NotI, and ligated with the 1.0 kb NotI(vector)/SpeI(vector) fragment from pHPN 1 (E/ED)/pCR™2.1 to yield pHPN1(E/ED)/pBluescriptII KS(−). The pHPN1(E/ED)/pBluescriptII KS(−) was cleaved by NotI(vector) and NheI(5125), and ligated with the 1.2 kb NotI(vector)/NheI(5124) fragment from pHPN1(C/F)/pCR™2.1 to yield pHPN1(C/ED)/pBluescriptII KS(−). The pHPN1(C/ED)/pBluescriptII KS(−) was cleaved by NotI(vector) and XbaI(3120), and ligated with the 3.9 kb NotI(vector)/XbaI(3919) fragment from pHPN1(ST/D)/pZeoSV to yield pHPN1/pBluescriptII KS(−), containing the entire coding sequence for human PN1.

The 6.1 kb NotI(vector)/SpeI(vector) fragment from pHPN1/pBluescriptII KS(−) was ligated into the XbaI/NotI site of the tTA-dependent expression vector pAHygTet1 containing a CMV minimal promoter and 7 tet-operators under control of tTA and tetracycline (14) to yield finally the inducible expression plasmid pHPN1/pAHygTet1 carrying the human PN1 cDNA.

1-2. Construction of Expression Plasmids Containing the Human PN3 cDNA

Complementary DNA encoding the human PN3 was cloned from human heart poly(A)⁺ RNA (Clontech, Tokyo, Japan) by the RT-PCR method as described above. Primer pairs were synthetic 25-nucleotide oligomers as follows;

1-25 (ST; upstream primer) and 1289-1313 (B; downstream primer),

1279-1303 (A; upstream primer) and 3350-3374 (D; downstream primer),

3339-3363 (C; upstream primer) and 4131-4155 (F; downstream primer), and

4121-4145 (E; upstream primer) and 5850-5874 (ED; downstream primer).

The numbering of nucleotides is according to the sequence derived from GenBank (accession number; AF 117907). The reverse transcriptase PCR products of the above primer pairs were ligated into pCR™ 2.1 vector or pCR4Blunt-TOPO vector (Invitrogen, Carlsbad, Calif.) to yield pHPN3 (ST/B)/pCR™2.1, pHPN3 (A/D)/pCR4Blunt-TOPO, pHPN3 (C/F)/pCR™2.1 and pHPN3(E/ED)/pCR™2.1, respectively. The plasmid pBluescriptII SK(−) (Stratagene, La Jolla, Calif.) was cleaved by HindIII and XbaI, and ligated with the 1.3 kb HindIII(vector)/XbaI(vector) fragment from pHPN3(ST/B)/pCR™2.1 to yield pHPN3(ST/B)/pbluescriptII SK(−). The pHPN3(ST/B)/pBluescriptII SK(−) was cleaved by NheI(1293) and NsiI(vector), and ligated with the 2.1 kb NheI(1294)/Nsil(3358) fragment from pHPN3(A/D)/pCR4Blunt-TOPO to yield pHPN3(ST/D)/pBluescriptII SK(−). The pHPN3(C/F)/pCR™2.1 was cleaved by SpeI(vector) and XmaI(4136), and ligated with 1.7 kb XmaI(4137)/SpeI(vector) fragment from pHPN3(E/ED)/pCR™2.1 to yield pHPN3(C/ED)/pCR™2.1. The plasmid pBluescriptII SK(−) was cleaved by XhoI and SpeI, and ligated with the 2.5 kb XhoI(vector)/SpeI(vector) fragment from pHPN3(C/ED)/pCR™2.1 to yield pHPN3(C/ED)/pBluescriptII SK(−). The pHPN3(ST/D)/pBluescriptII SK(−) was cleaved by NsiI(3358) and NotI(vector), and ligated with 2.5 kb NsiI(3359)/NotI(vector) fragment from pHPN3(C/ED)/pBluescriptII SK(−) to yield pHPN3/pBluescriptII SK(−), containing the entire coding sequence for human PN3 cDNA.

The 5.9 kb KpnI(vector)/NotI(vector) fragment from pHPN3/pBluescriptII SK(−) was further ligated with the NotI/KpnI site of pcDNA3.1/Zeo(+) (Invitrogen, Carlsbad, Calif.) to yield pHPN3/pcDNA3.1/Zeo(+). The synthetic oligodeoxyribonucleotide primers 5′-CATACAGGATGAAAAGAT GG-3′ (SEQ ID NO: 1) and 5′-AATTCCATCTTTTCATCCTGTATGGTAC-3′ (SEQ ID NO:2) were annealed to obtain the double-stranded DNA fragment, which contains the sequence of the 5′-untranslated region of human PN1 cDNA, nucleotide residues 34-48 derived from GenBank (accession number X82835), and the translation initiation site of human PN3 cDNA. The resulting fragment generated KpnI and EcoRI sites at the 5′- and 3′-end, respectively. This synthesized DNA was ligated with the EcoRI(5)/KpnI(vector) site of pHPN3/pcDNA3.1 /Zeo(+) to yield pHPN3-5′UTRPN1/pcDNA3.1/Zeo(+).

The pAHygTet1 vector was cleaved by XhoI, treated with T4 DNA polymerase (blunt-ended), and cleaved by NotI. The pHPN3-5′UTRPN1/pcDNA3.1/Zeo(+) was cleaved by KpnI, blunt-ended, and cleaved by NotI. The resulting 5.9 kb blunt-end(vector)/NotI(vector) fragment from pHPN3-5′UTRPN1/pcDNA3.1/Zeo(+) was ligated with the NotI/blunt-end site of pAHygTet1 to yield the inducible expression plasmid pHPN3-5′UTRPN1/pAHygTet1 carrying the human PN3 cDNA.

All the reverse transcriptase PCR products were sequenced by dideoxy chain termination method.

2. Isolation of Stable Transfectants

Chinese hamster ovary (CHO) cells were cotransfected with plasmids pHPN1/pAHygTet1 and PUHD15-1NEO, and pHPN3-5′UTRPN1/pAHygTet1 and pUHD15-1_{NEO}, by electroporation and LipofectAMINE PLUS™ (Life Technologies, Rockville, Md.), respectively. The cells were selected in α-Minimum Essential Medium containing 10% fetal bovine serum, 100 μg/ml hygromycin, 500 μg/ml G418, and 2 μg/ml tetracycline.

3. Northern blot analysis

To analyze the inducible expression of human PN1 and PN3 sodium channels, northern blot analysis was carried out, essentially as previously described (Nishimura S, et al. (1993) FEBS Lett., 324, 283-286). Total RNA (20 μg) from recombinant cell lines was incubated in 15 mM sodium phosphate buffer (pH 7.0) containing 9% glyoxal and 75% dimethylsulfoxide, and then fractionated by electrophoresis through a 1.5% agarose gel containing 10 mM sodium phosphate buffer (pH 7.0). The fractionated RNA was transferred to a Biodyne nylon membrane (Pall BioSupport, East Hills, N.Y.). After baking at 80° C. for 2 hours, the filter was prehybridized at 42° C. for 2 hours in 50 mM sodium phosphate buffer (pH 6.5) containing 5× SSC, 5× Denhardt's solution, 0.1% SDS, 0.25 mg/ml denatured herring sperm DNA and 50% formamide. The filter was hybridized at 42° C. for 16 hours with hybridization probes. The probes used were the HindIII(779)/HindIII(3600) fragment and the EcoRI(vector)/EcoRI(vector) fragment excised from plasmids pHPN1/pAHygTet1 and pHPN3(A/D)/pCR4Blunt-TOPO, respectively, and labeled by Oligolabelling kit (Pharmacia Biotech, Uppsala, Sweden) with [α-³²P]dCTP. Autoradiography was performed at −80° C. for 16 hours with an intensifying screen.

4. Electrophysiology

Na⁺currents were recorded at room temperature (22-25° C.) using whole-cell mode of the patch clamp with an EPC-9 Patch clamp amplifier (HEKA, Lambrecht, Germany). Patch pipettes were made from aluminosilicate glass (1.6 mm outer diameter, 1.2 mm inner diameter, Hilgenberg, Malsfeld, Germany). The patch electrodes were fire-polished. Pipette resistance ranged from 1 to 1.5 MΩ when filled with the pipette solutions described below. The series resistance was electronically compensated to greater than 80% and both the leakage and the remaining capacitance were subtracted by −P/6 method. The external solution contained (in mM): 135 NaCl, 4 KCl, 1.5 CaCl₂, 1 MgCl₂, 10 glucose and 10 HEPES (pH 7.4 with NaOH). The pipette solution contained (in mM): 70 CsF, 60 CsCl, 12 NaF, 5 HEPES, 0.1 BAPTA, 2 ATP, and 2 GTP (pH 7.4 with CsOH). Application of compounds was made by a modified “Y-tube” method. Currents were digitized at 100 kHz after low pass filtering at 10 kHz (−3 dB). Data were collected and analyzed using the Pulse and PulseFit software (HEKA, Lambrecht, Germany) and Igor Pro (WaveMetrics, Lake Oswego, Oreg.). The parameters for voltage dependence of activation were estimated from the current-voltage relation based on the equation, I=Gₘₐₓ×(V−Vᵣₑᵥ)/(1+exp(−(V−Vₘ)/aₘ)), where I is the peak current amplitude, Gₘₐₓ the maximum conductance, V the test potential, Vᵣₑᵥ the reversal potential, Vₘ the midpoint of activation, and am a slope factor. The dose-response curve was fitted with the Hill's equation, I=Iₘₐₓ×1/(1+(c/IC₅₀)ⁿ), where IC₅₀ is the concentration to block 50% of channels, and n is the Hill's coefficient.

### Results

1. Cloning of cDNAs Encoding the Human PN1 and PN3 Sodium Channels

Using the RT-PCR cloning strategy, we obtained the human PN1 and PN3 cDNAs from adrenal gland and heart poly(A)⁺ RNAs, respectively. The amino-acid sequence of the clones obtained showed identity with human PN1 (5), and PN3 (Rabert D K, et al. (1998) Pain, 78, 107-114). In the PN1 clone, silent mutations of the nucleotide-sequence were recognized at position 222 (A→G), 492 (G→A), 978 (A→G), 1314 (A→G), 1335 (T→A) and 4806 (T→C). In the PN3 clone, the mutations were also detected at position 3393 (C→G) and 5205 (T→C).

2. Establishment of Stable Transfectants Expressing the Human PN1 and PN3 Sodium Channels

There are no reports on the establishment of stable cell lines expressing human sodium channels due to difficulties in constitutive expression under physiologically unregulated conditions. To overcome this problem in the art, we adopted the use of inducible expression systems for the generation of stable transfectants expressing the peripheral nerve type PN 1 and PN3 sodium channels.

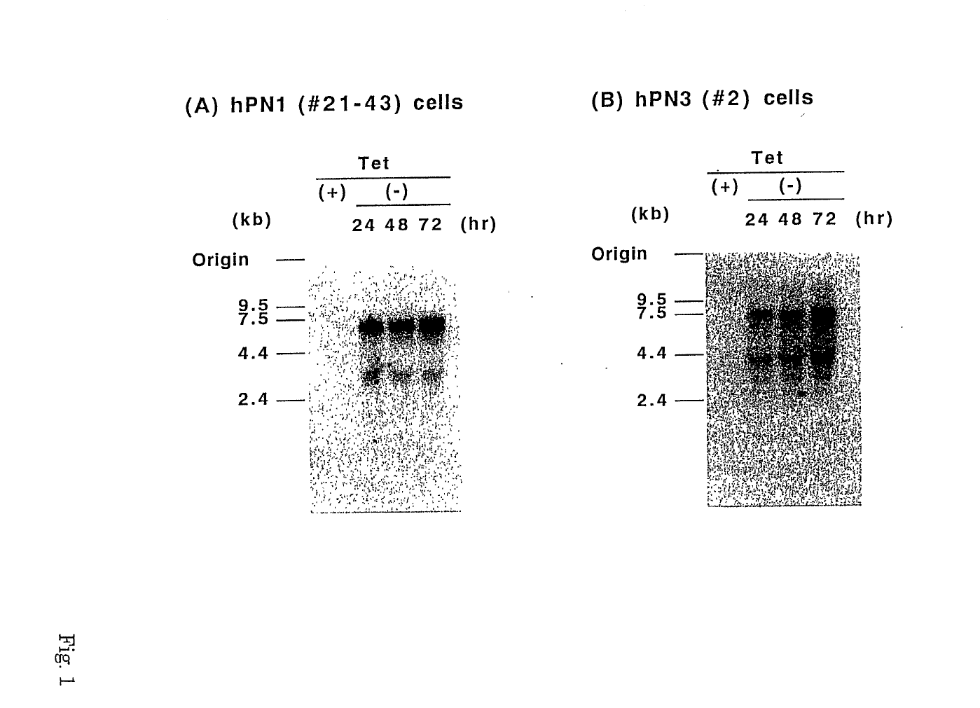

Stably transfected cell lines expressing the human PN1 and PN3 sodium channels hPN1(#21-43) and hPN3(#2), respectively, were established in CHO cells using a Tet-off inducible expression system. The expression of human PN1 and PN3 sodium channels was investigated by northern blot analysis (FIG. 1). In both transfectants, a strong signal with a size of approximately 7.5 kb was detected 24 hours after removal of tetracycline from the culture medium, but not in its presence. The hybridizable RNA species correspond to the expected size containing the cDNA for human PN1 or PN3. Their expression levels were gradually increased on the second and third day after tetracycline removal.

3. Electrophysiological Characterization of the Transfectants

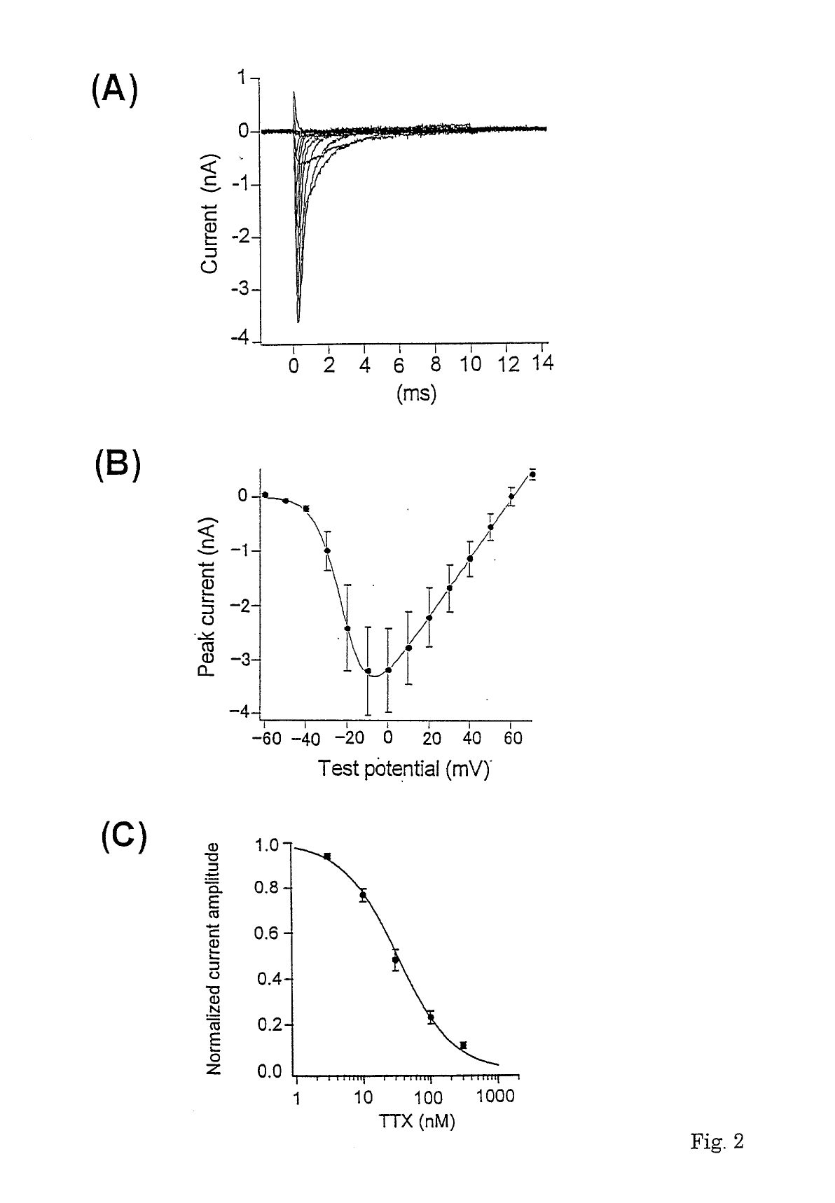

Whole-cell currents were examined to confirm the presence of functional voltage-gated sodium channels in the recombinant cell lines on the second and third day after tetracycline removal from the culture medium. In hPN1 (#21-43) cells, rapidly activating and inactivating inward currents were recorded (FIG. 2A). PN1 currents were activated at potential −40 mV and maximal currents were observed at −10 mV (FIG. 2B). The average peak current amplitude was 3.2±0.8 nA (mean±SE, n=7), and the average current density was 0.32+0.07 nA/pF. Values of the half maximal activation (V_{½}) and slope factor were −21.3±1.5 mV and 6.3±0.5 mV (n=7), respectively. The PN1 current was blocked by TTX with an IC₅₀ of 32.6±5.3 nM (Hill's coefficient 1.06±0.05; n=5) (FIG. 2C).

It is important to note that the cell line hPN1 (#21-43) was functionally stable over a period of at least six months (data not shown).

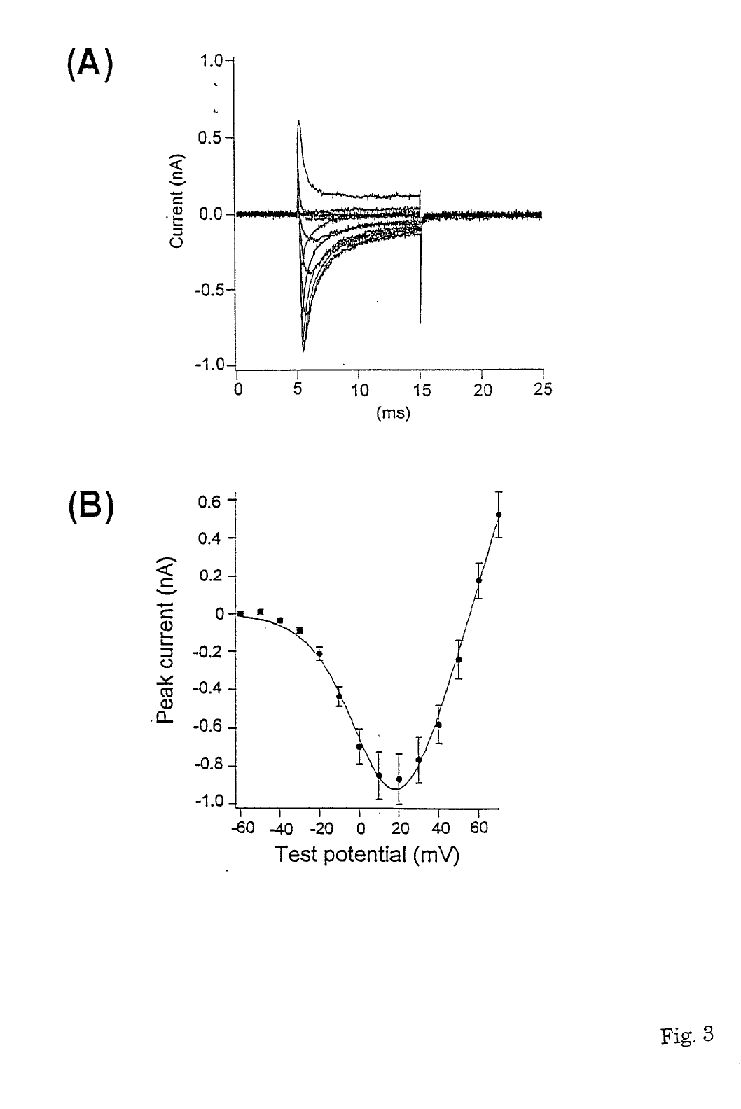

The PN3-expressing hPN3 (#2) cells produced an inward current that inactivated slowly compared to PN1, even in the presence of 1 μM TTX (FIG. 3A). The threshold for channel activation was −20 mV and maximal currents were observed at +20 mV (FIG. 3B). The average peak current amplitude was 0.87±0.13 nA (n=12), and the average current density was 0.055±0.012 nA/pF. The V_{½} and slope parameters for PN3 were 2.50±1.58 mV and 11.3±0.5 mV (n=12), respectively. The functional expression of human PN3 sodium channel in this cell line did not change after at least eight cell passages.

Although CHO cells are known to have endogenous Na⁺ channel activity which is TTX-sensitive, the peak current amplitude of the endogenous Na⁺ channel activity was rarely larger than 0.2 nA, indicating that the major portion of the observed currents were mediated by the recombinant Na⁺ channels.

What is claimed is:

1. A cell line comprising a recombinant nucleic acid, wherein said recombinant nucleic acid comprises one or more nucleotide sequences encoding one or more peripheral nerve (PN) sodium channels, wherein said PN sodium channel is stably expressed on the surface of said cell line.

2. The cell line according to claim 1, wherein said sodium channel comprises the peripheral nerve type sodium channel 1 (PN1).

3. The cell line according to claim 1, wherein said sodium channel comprises the peripheral nerve type sodium channel 3 (PN3).

4. The cell line according to claim 2, wherein said sodium channel further comprises the peripheral nerve type sodium channel 3 (PN3).

5. The cell line according to any one of claims 1 to 4, wherein said recombinant nucleic acid comprises a nucleotide sequence as in Genbank accession number X82835 or a functional derivative or a fragment thereof.

6. The cell line according to any one of claims 1 to 4, said recombinant nucleic acid comprises a nucleotide sequence as in Genbank accession number AFI 17907 or a functional derivative or a fragment thereof.

7. The cell line according to claim 5, wherein said recombinant nucleic acid further comprises a nucleotide sequence as in Genbank accession number AF117907 or a functional derivative or a fragment thereof.

8. The cell line according to any one of claims 1 to 4, wherein said peripheral nerve sodium channel is human.

9. The cell line according to any one of claims 1 to 4 which is derived from a CHO cell line.

10. The cell line according to claim 5 which is derived from a CHO cell line.

11. The cell line according to claim 6 which is derived from a CHO cell line.

12. The cell line according to claim 7 which is derived from a CHO cell line.

13. A CHO cell line comprising a recombinant nucleic acid, wherein said recombinant nucleic acid comprises a nucleotide sequence encoding human peripheral nerve type 1 (PN1) sodium channel, wherein said human PN1 sodium channel is stably expressed on the surface of said cell line.

14. A CHO cell line comprising a recombinant nucleic acid, wherein said recombinant nucleic acid comprises a nucleotide sequence encoding human peripheral nerve type 3 (PN3) sodium channel, wherein said human PN3 sodium channel is stably expressed on the surface of said cell line.

15. A nucleic acid comprising one or more nucleotide sequences encoding a recombinant human peripheral nerve (PN) sodium channel or recombinant human PN sodium channels.

16. The nucleic acid according to claim 15, wherein said nucleotide sequence comprises the sequence in Genbank accession number X82835 or a functional derivative or a fragment thereof or a variant due to the degenerate code.

17. The nucleic acid according to claim 15, wherein said nucleotide sequence comprises the sequence in Genbank accession number AF117907 or a functional derivative or a fragment thereof or a variant due to the degenerate code.

18. A method for preparing a cell line according to any one of claims 1 to 4, comprising:
a) constructing an expression vector which comprises a nucleic acid encoding a peripheral nerve (PN) sodium channel;
b) transfecting a suitable cell line with said expression vector; and
c) screening said cell line for stable transfection employing a suitable selection agent.

19. A method for preparing a cell line according to claim 5, comprising:
a) constructing an expression vector which comprises a nucleic acid encoding a peripheral nerve (PN) sodium channel;
b) transfecting a suitable cell line with said expression vector; and
c) screening said cell line for stable transfection employing a suitable selection agent.

20. A method for preparing a cell line according to claim 6, comprising:
a) constructing an expression vector which comprises a nucleic acid encoding a peripheral nerve (PN) sodium channel;
b) transfecting a suitable cell line with said expression vector; and
c) screening said cell line for stable transfection employing a suitable selection agent.

21. A method for preparing a cell line according to claim 7, comprising:
a) constructing an expression vector which comprises a nucleic acid encoding a peripheral nerve (PN) sodium channel;
b) transfecting a suitable cell line with said expression vector; and
c) screening said cell line for stable transfection employing a suitable selection agent.

22. The method for preparing a cell line according to claim 18, wherein said cell line is a CHO cell line.

23. The method according to claim 18, wherein said cell line is co-transfected with two or more of said expression vectors each of which comprises a nucleic acid specific for a different peripheral nerve (PN) sodium channel.

24. The method according to claim 22, wherein said cell line is co-transfected with two or more of said expression vectors each of which comprises a nucleic acid specific for a different peripheral nerve (PN) sodium channel.

25. A method for identifying an agonist, antagonist or modulator of a peripheral nerve (PN) sodium channel comprising:
a) incubating a cell line according to any one of claims 1 to 4 with a test substance;
b) measuring a membrane current of said cell line after incubation with test substance;
c) incubating said cell line with a known control or in the absence of said test substance;
d) measuring a membrane current of said cell line after incubation with a known control or in the absence of said test substance; and
e) comparing said membrane current in step (b) with said membrane current obtained in step (d).

26. A method for identifying an agonist, antagonist or modulator of a peripheral nerve (PN) sodium channel comprising:
a) incubating a cell line according to claim 5 with a test substance;
b) measuring a membrane current of said cell line after incubation with test substance;
c) incubating said cell line with a known control or in the absence of said test substance;
d) measuring a membrane current of said cell line after incubation with a known control or in the absence of said test substance; and
e) comparing said membrane current in step (b) with said membrane current obtained in step (d).

27. A method for identifying an agonist, antagonist or modulator of a peripheral nerve (PN) sodium channel comprising:
a) incubating a cell line according to claim 6 with a test substance;
b) measuring a membrane current of said cell line after incubation with test substance;
c) incubating said cell line with a known control or in the absence of said test substance;
d) measuring a membrane current of said cell line after incubation with a known control or in the absence of said test substance; and
e) comparing said membrane current in step (b) with said membrane current obtained in step (d).

28. A method for identifying an agonist, antagonist or modulator of a peripheral nerve (PN) sodium channel comprising:
a) incubating a cell line according to claim 7 with a test substance;
b) measuring a membrane current of said cell line after incubation with test substance;
c) incubating said cell line with a known control or in the absence of said test substance;
d) measuring a membrane current of said cell line after incubation with a known control or in the absence of said test substance; and
e) comparing said membrane current in step (b) with said membrane current obtained in step (d).

29. The method according to claim 25 wherein said cell line is derived from a CHO cell line.

30. A method for identifying an agonist, antagonist or modulator of a peripheral nerve (PN) sodium channel comprising:
a) incubating a cell line according to any one of claims 1 to 4 with a known agonist which is coupled to a reporter;
b) incubating said cell line with a test substance; and
c) measuring the displacement of the agonist coupled to the reporter by the test substance.

31. A method for identifying an agonist, antagonist or modulator of a peripheral nerve (PN) sodium channel comprising:
a) incubating a cell line according to claim 5 with a known agonist which is coupled to a reporter;
b) incubating said cell line with a test substance; and
c) measuring the displacement of the agonist coupled to the reporter by the test substance.

32. A method for identifying an agonist, antagonist or modulator of a peripheral nerve (PN) sodium channel comprising:
a) incubating a cell line according to claim 6 with a known agonist which is coupled to a reporter;
b) incubating said cell line with a test substance; and
c) measuring the displacement of the agonist coupled to the reporter by the test substance.

33. A method for identifying an agonist, antagonist or modulator of a peripheral nerve (PN) sodium channel comprising:
a) incubating a cell line according to claim 7 with a known agonist which is coupled to a reporter;
b) incubating said cell line with a test substance; and
c) measuring the displacement of the agonist coupled to the reporter by the test substance.

34. The method according to claim 25, wherein said method is a high throughput screening assay (HTS).

35. The method according to claim 30, wherein said method is a high throughput screening assay (HTS).

36. The method according to claim 30, wherein said reporter is a fluorescent molecule or a radioisotope.

37. The method according to any one of claims 31 to 33, wherein said reporter is a fluorescent molecule or a radioisotope.

38. An agonist, antagonist or modulator of a peripheral nerve (PN) sodium channel identifiable according to the method of claim 25.

39. An agonist, antagonist or modulator of a peripheral nerve (PN) sodium channel identifiable according to the method of claim 30.

40. The agonist, antagonist or modulator according to claim 38, wherein said agonist, antagonist or modulator is not selected from the group of lidocaine, mexiletine and TTX.

41. The agonist, antagonist or modulator according to claim 39, wherein said agonist, antagonist or modulator is not selected from the group of lidocaine, mexiletine and TTX.

42. A pharmaceutical composition comprising an agonist, antagonist or modulator of a peripheral nerve (PN) sodium channel according to claim 38; and a pharmaceutically acceptable carrier.

43. A pharmaceutical composition comprising an agonist, antagonist or modulator of a peripheral nerve (PN) sodium channel according to claim 39; and a pharmaceutically acceptable carrier.
